(19) United States
(12) Patent Application Publication
(10) Pub. No.: US 2021/0027897 A1
(43) Pub. Date: Jan. 28, 2021

(54) SKIN HEALTH TRACKER

(71) Applicant: DERMALA INC., San Diego, CA (US)

(72) Inventors: Lada Rasochova, Del Mar, CA (US); Alexander Abdel Alim, Rancho Santa Fe, CA (US)

(73) Assignee: DERMALA INC., San Diego, CA (US)

(21) Appl. No.: 17/042,885

(22) PCT Filed: Mar. 26, 2019
(86) PCT No.: PCT/US19/24130

§ 371 Date: Sep. 28, 2020

Related U.S. Application Data
(60) Provisional application No. 62/648,307, filed on Mar. 26, 2018.

(51) Int. Cl. G16H 50/70; G16H 10/60; G16H 50/20; G16H 70/20; G16H 50/30; G16H 50/50; G16H 10/40; G16H 10/20; G16H 70/60; G16H 20/10; G16H 20/70; G06T 7/00; A61B 5/00; A63F 13/65

(52) U.S. Cl. CPC G16H 50/70; A61B 2576/02; G16H 50/20; G16H 70/20; G16H 50/30; G16H 50/50; G16H 10/40; G16H 10/20; G16H 70/60; G16H 20/10; G16H 20/70; G06T 7/0016; A61B 5/445; A61B 5/0077; A61B 5/7267; A61B 5/0013; A63F 13/65; G06T 2207/30088; G16H 10/60

(57) ABSTRACT

An artificial intelligence-supported mobile or internet application is disclosed that receives and analyzes image of skin and associated information. Algorithms and learning techniques are applied to generate a treatment plan for the user. The treatment plan is continuously monitored to determine the effectiveness of the treatment. New factors may be identified as variables that impact skin health by using the algorithms and learning techniques disclosed herein.

### CROSS-REFERENCE TO RELATED APPLICATIONS

This application claims the benefit of PCT Application No. PCT/US19/24130, Filed on Mar. 26, 2019, which claims the benefit of U.S. Provisional Application Ser. No. 62/648,307 filed on Mar. 26, 2018.

### STATEMENT REGARDING FEDERALLY SPONSORED RESEARCH OR DEVELOPMENT

Not Applicable.

### FIELD

This invention relates to a computer system and methods for analyzing, treating, and tracking skin disorders. Specifically, this invention uses novel techniques to diagnose and classify skin disorders, personalize treatment, and track treatment outcome.

### INTRODUCTION

Traditionally, individuals seeking treatment of their skin disorders would have to visit a dermatologist in person. The dermatologist, based on the current state of the patients' skin, would recommend a treatment plan they believe would improve the condition of the patient's skin disorder. The patient would then follow that treatment plan for a month, or another predetermined amount of time, and then return for a visual analysis. The dermatologist would then adjust the treatment plan according to patient's skin condition in the next appointment. This cycle may continue for months or years. Patients would often experience frustration and lack of guidance as they experience real or perceived lack of improvement.

While this method of treating patients works to some capacity, the outcomes are not always desirable, efficient, and objectively evaluated. Specifically, patients may lose motivation or not understand the life cycle of their condition or timeframe for improvement. Additionally, patients do not receive adequate education or timely feedback to change or improve their treatment plan.

### SUMMARY

The present teachings include a method of treating and monitoring skin conditions for each user of one or more users. The method involves the following steps: (1) receiving medical information of each user of the one or more users; (2) receiving demographic information of each user of the one or more users; (3) receiving one or more images of each user of the one or more users, wherein the one or more images depict one or more affected skin areas; (4) sending the medical information for each user of the one or more users, the demographic information for each user of the one or more users, and the one or more images for each user of the one or more users to a server; (5) generating an artificial intelligence (AI)-supported model to yield a profile for each user of the one or more users, at the server, wherein the profile for each of the one or more users comprises the medical information, the demographic information, and the one or more images; (6) extracting contents from a database using the AI-supported model, wherein the contents from the database comprise established treatment plans, medical findings, and effects of local environments on skin regions; (7) applying the AI-supported model on the profile for each of the one or more users and the contents from the database, and thereby: (a) establishing a baseline for each user of the one or more users, and (b) devising a customized plan for each user of the one or more users; (8) receiving subsequent medical information for each user of the one or more users and subsequent one or more images of the one or more users; (9) comparing the subsequent medical information for each user of the one or more users and subsequent one or more images of the one or more users to the medical information for each user of the one or more users, the demographic information for each user of the one or more users, and the one or more images for each user of the one or more users, and thereby tracking progress of the customized plan of each user of the one or more users; and (10) outputting effectiveness of the customized treatment plan for each user of the one or more users.

In accordance with a further aspect of the method, the medical information and the subsequent medical information for each user of the one or more users comprise a type of acne and location of the type of acne.

In accordance with yet another aspect of the method, the demographic information and the subsequent medical information for each user of the one or more users comprise age, gender, and location for each user of the one or more users.

In accordance with yet another aspect of the method, generating the AI-based model comprises: treating the one or more images and the subsequent one or more images of each user of the one or more users and the contents as a first data stream; and treating the medical information and the subsequent medical information of each user of the one or more users and the demographic information of each of the one of the one or more users as a second data stream.

In accordance with yet another aspect of the method, tracking progress of the customized plan for each user of the one or more users comprises: identifying a first set of factors impacting the one or more regions of the skin; and adding a second set of factors to the database.

In accordance with yet another aspect of the method, the method also involves: requesting biological test results; and integrating the biological test results into the database.

In accordance with yet another aspect of the method, devising the customized treatment plan for each user of the one or more users, comprises: receiving inputs to inquiries from each user of the one or more users; comparing the inputs to the inquiries from each user of the one or more users; identifying similarities and differences between the inputs to the inquiries from each user of the one or more users; grouping the one or more users as at least a first set and a second set, based on the similarities and differences.

In accordance with yet another aspect of the method, grouping the one or more users as at least the first set and the second set comprises: devising a customized treatment plan for the first set; and devising a customized treatment plan for the second set.

In accordance with yet another aspect of the method, the method also involves generating a virtual game based on the AI-based model.

In accordance with yet another aspect of the method involves, the biological tests are used to obtain microbiome, genome, epigenome, and pH data.

In accordance with yet another aspect of the method, outputting the effectiveness of the customized treatment plan for each user of the one or more users, comprises: determining if the effectiveness of the customized treatment plan for each user of the one or more users meets a threshold level; validating a customized plan as effective if the effectiveness is above the threshold level; and modifying the customized plan if the effectiveness is below the threshold level.

The present teachings include a computer program product. When executing computer executable code embodied in a non-transitory computer readable medium on one or more computing devices, the computer program product performs steps. The steps involve: (1) receiving medical information of each user of the one or more users; (2) receiving demographic information of each user of the one or more users; (3) receiving one or more images of each user of the one or more users, wherein the one or more images depict one or more affected skin areas; (4) sending the medical information for each user of the one or more users, the demographic information for each user of the one or more users, and the one or more images for each user of the one or more users to a server; (5) generating an artificial intelligence (AI)-supported model to yield a profile for each user of the one or more users, at the server, wherein the profile for each of the one or more users comprises the medical information, the demographic information, and the one or more images; (6) extracting contents from a database using the AI-supported model, wherein the contents from the database comprise established treatment plans, medical findings, and effects of local environments on skin regions; (7) applying the AI-supported model on the profile for each of the one or more users and the contents from the database, and thereby: (a) establishing a baseline for each user of the one or more users, and (b) devising a customized plan for each user of the one or more users; (8) receiving subsequent medical information for each user of the one or more users and subsequent one or more images of the one or more users; (9) comparing the subsequent medical information for each user of the one or more users and subsequent one or more images of the one or more users to the medical information for each user of the one or more users, the demographic information for each user of the one or more users, and the one or more images for each user of the one or more users, and thereby tracking progress of the customized plan of each user of the one or more users; and (10) outputting effectiveness of the customized treatment plan for each user of the one or more users.

In accordance with a further aspect of the computer program product, the medical information and the subsequent medical information for each user of the one or more users comprise a type of acne and location of the type of acne.

In accordance with a yet another aspect of the computer program product, the demographic information and the subsequent medical information for each user of the one or more users comprise age, gender, and location for each user of the one or more users.

In accordance with a yet another aspect of the computer program product, generating the AI-based model comprises: treating the one or more images and the subsequent one or more images of each user of the one or more users and the contents as a first data stream; and treating the medical information and the subsequent medical information of each user of the one or more users and the demographic information of each of the one of the one or more users as a second data stream.

In accordance with a yet another aspect of the computer program product, tracking progress of the customized plan for each user of the one or more users comprises: identifying a first set of factors impacting the one or more regions of the skin; and adding a second set of factors to the database.

The present teachings include a system. The system includes a server and a computing device in communication with the server over a network. The computing device includes a processor and memory. Computer executable code in the memory is configured to perform steps. The steps involve: (1) receiving medical information of each user of the one or more users; (2) receiving demographic information of each user of the one or more users; (3) receiving one or more images of each user of the one or more users, wherein the one or more images depict one or more affected skin areas; (4) sending the medical information for each user of the one or more users, the demographic information for each user of the one or more users, and the one or more images for each user of the one or more users to a server; (5) generating an artificial intelligence (AI)-supported model to yield a profile for each user of the one or more users, at the server, wherein the profile for each of the one or more users comprises the medical information, the demographic information, and the one or more images; (6) extracting contents from a database using the AI-supported model, wherein the contents from the database comprise established treatment plans, medical findings, and effects of local environments on skin regions; (7) applying the AI-supported model on the profile for each of the one or more users and the contents from the database, and thereby: (a) establishing a baseline for each user of the one or more users, and (b) devising a customized plan for each user of the one or more users; (8) receiving subsequent medical information for each user of the one or more users and subsequent one or more images of the one or more users; (9) comparing the subsequent medical information for each user of the one or more users and subsequent one or more images of the one or more users to the medical information for each user of the one or more users, the demographic information for each user of the one or more users, and the one or more images for each user of the one or more users, and thereby tracking progress of the customized plan of each user of the one or more users; and (10) outputting effectiveness of the customized treatment plan for each user of the one or more users.

In accordance with a further aspect of the system, the medical information and the subsequent medical information for each user of the one or more users comprise a type of acne and location of the type of acne.

In accordance with a yet another aspect of the system, the demographic information and the subsequent medical information for each user of the one or more users comprise age, gender, and location for each user of the one or more users.

In accordance with a yet another aspect of the system, generating the AI-based model comprises: treating the one or more images and the subsequent one or more images of each user of the one or more users and the contents as a first data stream; and treating the medical information and the subsequent medical information of each user of the one or more users and the demographic information of each of the one of the one or more users as a second data stream.

These and other features, aspects, and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1 depicts an exemplary computing environment for artificial intelligence-supported (AI-supported) digital application to analyze the skin health of a user.

FIG. 2 depicts a flowchart summarizing the operations performed by the AI-supported digital application.

FIG. 3 depicts a user registration process for the AI-supported digital application, as displayed in a graphical user interface (GUI).

FIG. 4 depicts user logs, progress tracking, and detail views for the AI-supported digital application, as displayed in a GUI.

FIG. 5 depicts an example of the analytics performed by the AI-supported digital application, as displayed in a GUI.

FIG. 6 depicts an operational loop performed by the AI-supported digital application.

FIG. 7 depicts inquires used by the AI-supported digital application.

FIG. 8 depicts an example of improved clinical outcomes for a user using the treatment plan proposed by AI-supported digital application.

### DETAILED DESCRIPTION

### Abbreviations and Definitions

To facilitate understanding of the invention, a number of terms and abbreviations as used herein are defined below.

Dermatology: As used herein, the term “dermatology” refers to the branch of medicine dealing with the function and treatment of medical conditions and disorders directed to skin, nails, and hair. The medical implications of dermatological conditions (e.g., lupus, bullous pemphigoid, acne, and eczema) include, but are not limited to: undesired cosmetic appearance of the skin, nails, and hair; cancer; and skin infections.

Acne: As used herein, the term “acne” (which is also known as acne vulgaris) refers to a long-term skin disease that occurs when dead skins cells and oil from the skin clog hair follicle.

Eczema: As used herein, the term “eczema” (which is also known as dermatitis) refers to inflammation of the skin, characterized by itchiness, red skin, and rashes.

Artificial intelligence (AI): As used herein, the term “AI” (which is also referred to machine learning (ML)) refers to a computing system that learns from experiences, make adjustments based on the experiences, and perform human-like tasks. The learning may be unsupervised (i.e., the ability to find patterns in a stream of input without requiring a human to initially label the inputs) or supervised (i.e., the ability to find patterns in a stream of input requiring a human to initially label the inputs).

Microbiome: As used herein, the term “microbiome” refers to the collective genomes of microorganisms and viruses residing in an environment or the microorganisms and viruses themselves. The microorganisms and viruses may inhabit the skin of human or other members of the animal kingdom.

Genome: As used herein, the term “genome” refers to genes, noncoding deoxyribonucleic acid (DNA), mitochondrial DNA, and chloroplast DNA.

Epigenome: As used herein, the term “epigenome” refers to a record of chemical changes to DNA and histone proteins of an organism. Alterations in the epigenome by environmental conditions and chemical changes can result in alterations in the structure of chromatin and the function of the genome.

Metabolome: As used herein, the term “metabolome” refers to a complete set of small-molecule chemical found in a cell, a cellular organelle, an organ, a tissue, a tissue extract, a biofluid, or an entire organism. The small-molecule may be endogenous chemicals naturally produced by an organism or exogenous chemicals not naturally produced by an organism.

Data: As used herein, the term “data” refers to information collected and processed by the AI-techniques described herein. Data can also include a collection of information, digital text, handwriting, numerical tables, and the like.

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” “substantially,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms.

In general, the devices, systems, and methods discussed herein may utilize image and text analysis, which may be automated through the use of various hardware and software as described herein. The image and text analysis techniques discussed herein may thus be used for the detection of non-dermatological situations and categorizing images and other data to assist in providing and monitoring treatment plans.

The present invention is directed to a computer system and methods for optimizing treatment of skin conditions and disorders, based on treatment progress; behavior; diet; user engagement; gut microbiome and metabolome; skin microbiome and metabolome; photo analysis and tracking; and other inputs and data generated by or collected from the user. An AI-supported digital application determines various data inputs that aide in the skin diagnosis and prognosis to efficiently evaluate a user's skin. When the user begins using the AI-supported digital application, the AI-supported digital application prompts the user to answer an initial set of questions, deriving from a designated list of question, that help in the classification and understanding of a user's skin health. The initial set of questions may pertain, without limitation, to demographic information (e.g., age, gender, and location) and medical information (e.g., severity of acne, acne lesion count, type of acne, and previous treatment history). Questions beside the initial set of question also derive from the designated list of questions. Some or all of these additional questions may be presented to the user. At future points in time, the questions presented to the user may vary from the initial set of questions, based on responses to the given questions. The AI-supported digital application may devise new questions, which are different from the questions in the designated list of questions, to gather more detail on the responses to the given questions.

The user may also be prompted or instructed to take and upload a photo of the user's affected skin area. A user may also be provided with a microbiome sequencing kit for taking samples of their gut and skin microbiome. Upon the AI-supported digital application receiving sequencing results of the user's skin and gut microbiome, the user's skin condition from a bacterial level may be better understood. The combination of the microbiome sequencing results and the user-generated data provide a baseline or starting point of a user's current skin health. If the AI-supported digital application determines the user has an identifiable skin condition for which a treatment regimen may be applied, a specifically selected treatment plan is then assigned to the user to aide in the rebalancing of the user's skin to correct the condition. Based on inputted responses to questions, such as type of and severity of acne, the AI-supported digital application devises a customized treatment plan that is suggested to the user as the initial treatment for the skin condition. Additionally, a virtual game may be generated by the AI-supported digital application to track the progress of customized treatment plans across a group of users. This allows the AI-supported digital application to identify similarities and differences among the group of users participating in the virtual game. In turn, new variables that impact skin health may be identified.

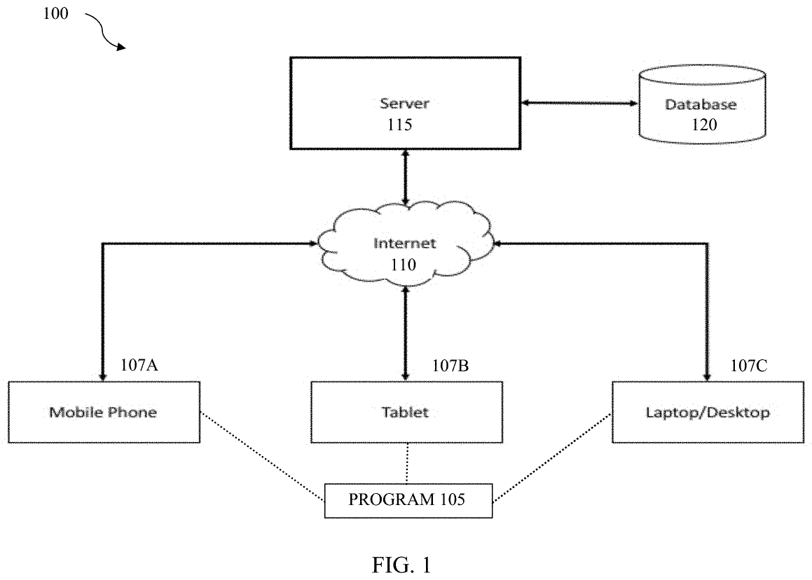

The systems and methods disclosed herein may be implemented via one or more components, systems, servers, appliances, other subcomponents, or distributed between such elements. When implemented as computing environment 100, such systems may include and/or involve, inter alia, components such as software modules, general-purpose CPU, RAM, etc., found in general-purpose computers. In implementations where the innovations reside on a server, such a server may include or involve components such as CPU, RAM, etc., such as those found in general-purpose computers. With respect to FIG. 1, program 105 resides in device 107, which is a computing device with a graphical user interface (not shown); a user input system (not shown), such as a mouse, keyboard, or touchpad; and camera components (not shown) that take direct photos and self-portrait images (e.g., “selfies”); and sensors (not shown) that detect shifts in gyrations, lighting, orientation, temperature, force, and so forth. In this example, device 107A is a mobile phone; device 107B is a tablet, and device 107C is a laptop/desktop. A user of devices 107A, 107B, and/or 107C connect to server 115 via internet 110, wherein the server 115 connects to database 120.

The internet 110 may include a communications path such as a wired or wireless network that uses a communications protocol and a data protocol, such as HTTP, HTTPS, HTML, JSON, or REST, to allow each of the devices 107A-C to interact with the server 115 and the database 120. The internet 110 may be a wired network, a wireless computer network, a wireless digital data network, a cellular wireless digital data network, or a combination of these networks that form a pathway each of the devices 107A-C, the server 115, and the database 120.

The internet 110 may also or instead include any data network(s) or internetwork(s) suitable for communicating data and control information among participants in the system 100. This may include public networks such as the Internet, private networks, and telecommunications networks such as the Public Switched Telephone Network or cellular networks using third generation cellular technology (e.g., 3G or IMT-2000), fourth generation cellular technology (e.g., 4G, LTE, MT-Advanced, E-UTRA, etc.) or WiMax-Advanced (IEEE 802.16m) and/or other technologies, as well as any of a variety of corporate area, metropolitan area, campus or other local area networks or enterprise networks, along with any switches, routers, hubs, gateways, and the like that might be used to carry data among users in the computing environment 100. The internet 110 may also include a combination of data networks, and need not be limited to a strictly public or private network.

The program 105 may be a mobile application on the device 107A or the device 107B, or an internet application on the device 107C. The program 105 may contain application programming interfaces (APIs) to communicatively connect to organized collections of data (e.g., the database 120) and a virtual private server used for cloud computing (e.g., the server 115). Database 120 may contain, but not is limited to, the following contents pertaining to dermatology and healthcare: established treatment plans that have demonstratively improved skin health (e.g., particular ointments for cases of severe acne for teenagers); medical findings (e.g., caustic acid burns impact the skin differently than caustic base burns); effects of local environments on skin regions on a personal level (e.g., acne flare-ups in a region due to increased stress levels due to pending exams) or a geographic level (e.g., reported cases of a high proportion of poison ivy species in a coastal plain correlated with rashes in the coastal plain); high resolution images of skin regions of the user and associated patient information of user (e.g., age, gender, race, natural hair color, residence, immediate location, and so forth). The program 105 is a digital application that may be supported by the techniques and models of AI. The program 105 may instruct the server 115 to receive information inputted into the device 107 and extract the contents from the database 120.

AI may perform the following functions on the information and contents in database 120 and server 115: (i) automatically discover the representations needed for feature detection or classification from raw data in the (i.e., feature learning); (ii) identify outliers (i.e., anomaly detection); (iii) make conclusion from observations (i.e., decision trees as a predictive model); (iv) discover relationships (i.e., associate rule learning); (v) create models; (vi) store findings in functions (i)-(iv) to the database 120 (e.g., NoSQL database); and (vii) create virtual games for a group of users to participate in, to derive new insights into dermatological health of an individual user or multiple users. The AI-supported models establish a baseline for each user, while using the information and contents in the server 115 and the database 120 as training sets to devise a treatment plan for user. Some of the AI-based models include, but not limited to, the following: Artificial Neural Networks (ANN); Support Vector Machines (SVM); Bayesian Networks; Deep Convolutional Network; Deconvolutional Network; Deep Convolutional Inverse Graphics Network; Generative Adversarial Network; Liquid State Machine Neural Network; Extreme Learning Machine; Neural Network; Echo State Network; Deep Residual Network; and Genetic Algorithms. This allows the program 105 to perform ensemble modeling so results and analysis of different models yield an optimal AI-supported digital application. For example, a user indicates his/her skin has acne, when it is actually a laceration. The program 105 sends the image taken by the user and the acne indication by the user to the server 115. Program 105 instructs the server 115 to apply analytics and correction factors to reconcile the image, which is actually a laceration; and change the incorrect indication of acne to a laceration.

The AI-capabilities of the program 105 may lead to personalization of dermatological treatment plans, based on numerous factors, but not limited to, demographic data, environmental data, skin data, and other health-related data (e.g., temperature and prior medication used for skin ailments). In the server 115, the program 105 receives responses to questions about the user's skin as an explicit data set, wherein the program 105 instructs a clustering algorithm to group the responses for the user based on the explicit data set. An image of a user's acne afflicted face is received by the server 115. Features of the image (e.g., regions of differing pigmentation, nature acne severity, number of acnes, regions of face afflicted by acne, and so forth) are extracted by the program 105 to generate explicit data in relation to the user's skin health. The program 105 may apply AI-supported models on the extracted features and generated explicit data in relation the user's skin heath to further classify and label the user.

The program 105 may use explicit data by prompting the user to self-report and self-diagnose, while harnessing implicit data from other available data streams. These data streams include image data. As the program 105 learns from the different data streams that are available, the program 105 assigns accurate weights to the data inputs, while more accurately estimating the expected output. This may allow the program 105 to find the best customized treatment plan forward for a user and further pinpointing a timeline until clear or improved skin. Treatment plan comprises suggested solution(s) that aim to improve the dermatological outcomes of a user or patient. The treatment plan may include, but are not limited to: treatment products and/or compositions (e.g., ingredient choice, active ingredient strength, and so forth); treatment regimen (e.g., how the treatment product and/or composition is applied, how often the treatment product and/or composition is applied, and so forth); and behavioral recommendations directed to avoiding stressful situations or other situations that may negatively impact skin health of the user of the program 105 (e.g., suggesting the user not operate a vehicle during rush hour, suggesting the user start working on a project that is due in a week to lessen the possibility of procrastinating, and so forth). The treatment products may include natural products (e.g., coconut oil, tea tree oil, apple cider vinegar, and aloe vera) and non-natural products (e.g., over-the-counter medications and prescribed medicine).

In turn, the program 105 may clinically benefit users and dermatological patients. More specifically, the program 105 may lead to the following benefits: (i) product personalization for treatment; (ii) optimization of product formulations (specific changes to treatment product composition); (iii) optimization of treatment regimen (specific changes to treatment product usage); (iv) prediction of treatment progression (timeline to results); and (v) identification of factors effecting disease and treatment progression.

Additionally, the systems and methods herein may be achieved via implementations with disparate or entirely different software, hardware and/or firmware components, beyond that set forth above. With regard to such other components (e.g., software, processing components, etc.) and/or computer-readable media associated with or embodying the present implementations, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the systems and methods may be achieved via or performed by logic and/or logic instructions including program modules, executed in association with such components or circuitry, for example. In general, program modules may include routines, programs, objects, components, data structures, etc., that perform particular tasks or implement particular instructions herein. The embodiments may also be practiced in the context of distributed software, computer, or circuit settings where circuitry is connected via communication buses, circuitry, or links. In distributed settings, control/instructions may occur from both local and remote computer storage media including memory storage devices.

The software, circuitry, and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, where media of any type herein does not include transitory media. Combinations of the any of the above are also included within the scope of computer readable media.

In the present description, the terms component, module, device, etc. may refer to any type of logical or functional software elements, circuits, blocks, and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays, or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software, and/or firmware. Moreover, the above-noted features and other aspects and principles of the innovations herein may be implemented in various environments. Such environments and related applications may be specially constructed for performing the various routines, processes, and/or operations according to the implementations described herein or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the implementations herein, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

Moreover, the above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

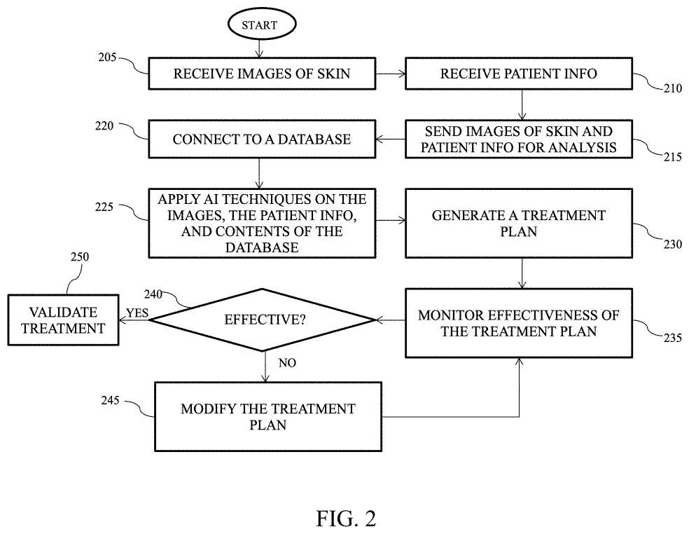

The operations of the flow chart in FIG. 2 may be performed by the AI-supported digital application program 105. A user activates and opens the program 105 in his or her version of the device 107.

In step 205, the program 105 receives image of the user's skin using the camera of the phone or images taken by another camera that is sent to the program 105.

In step 210, the program 105 receives patient information of the user. The patient information may be a series of questions directed to medical history and the skin or a series of demographic questions. These questions may be, but are not limited to:

What type of acne does the user have?

How many pimples does the user have per week, on average?

How often does the user have acne break outs?

What best describes skin type of the user?

Does the user have sensitive skin?

What are some of the Acne products previously taken by the user?

What are the types of makeup and other cosmetic products taken by the user?

What is the age of the user?

What is the gender of the user?

What is the racial background of the user?

What is the geographic location (ZIP code) of the user?

In step 215, the program 105 sends the images of the skin and the patient info for analysis at the server 115. In step 220, the program 105 connects to a database, such as the database 120.

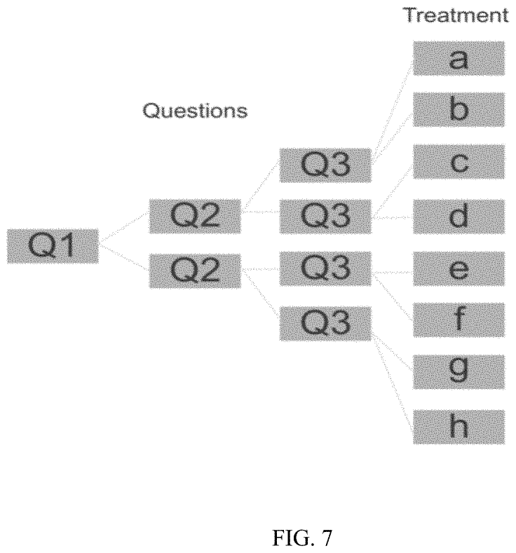

In step 225, the program 105 applies AI techniques on the images; the patient info, which includes inputs and responses to the questions or inquiries; and the contents of the database, as described with respect to the database 120. This leads to the user's initial classification into a group, which is described in more detail with respect to FIG. 7. The program 105 may create a profile for the user that compiles, but is not limited, to the following contents: the received and analyzed images; patient info; responses; and correlations or connections between disparate pieces of data (e.g., an image of ACNE and a response indicating exposure to a caustic chemical). Profiles for other users may be uploaded to the server 115 and stored in the database 120.

More specifically, the program 105 analyzes the images using machine learning and deep learning techniques. This may aid in improving the ability of the program 105 to make better decisions on the user's skin health when generating a treatment plan. As this is not explicitly given by the user, the image data is less biased and based on the skin's physical change over time. A convolutional neural network may be used for the architecture in computing environment 100 for accurate detection of acnes.

In conjunction with the user log data, the system will use computer vision techniques to analyze and supplement the user's explicit data with data generated from an uploaded selfie with each log. Data that could be abstracted from these images may further evolve and include skin color, skin oiliness, acne type, afflicted region, gender classification, likelihood of happiness vs. sadness, and so forth. For example, the program 105 may make assumptions on mental health and happiness based on the expression a user exhibited in an image. In conjunction with questions on anxiety, the expression of the user may provide insights into the user's mental state.

As the program 105 becomes more accurate, it may determine that certain questions or subsets of questions are no longer important as the computer vision methods deployed have become more accurate. Similar to a dermatologist recommending treatment regimen alterations based on viewing a user's skin, the program 105 may ultimately assume the same capabilities. Furthermore, the program 105 may become improved upon and refined by analyzing the profile of the user in comparison with other users.

Image data initially received from the first x number of users may be used to train the models applied by the program 105. Techniques have been deployed that require users to upload images of certain quality and fitting within certain constraints. For example, initial facial recognition technologies have been deployed to require images of a user's face that include the correct areas of face. This allows the program 105 to collect high-quality data on which it can be trained. These images may be labeled and preprocessed for use in training the model. As the model becomes exposed to more and more images, it may learn over time how to recognize different types of acnes (i.e. blackheads, whiteheads, pustules etc.). This ultimately means that the program 105 will be able to further classify the users based on the skin condition, such as the acne type; and derive characteristics of users (e.g., inclination of a user's skin to flare up during stressful times).

In step 230, the program 105 generates a treatment plan for the user, based on the classification of the user; skin condition; and derived characteristics. The treatment plan aims to be customized based on the information incorporated in the profile of the user. A monolith approach to dermatology is ineffective as location, genetic dispositions, stressors, and so forth impact individual users differently. User A and User B reside in the same, while they have differing levels of acne. The program 105 can account for other factors to treat the acne of User A and User B, besides the common location. Accordingly, the program 105 may devise a customized treatment plan for the user.

In step 235, the program 105 monitors the effectiveness of the treatment plan after the user's initial classification into a group. By uploading a photo and providing responses to a series of questions, users may follow up by logging their improvement or change in skin health. These questions and responses aid the program 105 in understanding how the user's health is responding to the treatment plan, as well as understand other variables in a user's regimen and lifestyle. Questions that a user could be asked are:
How has the user's skin health been since last log?
Did you the user apply any DERMALA® products on his or her skin?
If the user applied DERMALA® product, which product and how often?
How often did the user wash his or her face?
How oily has the user's skin been?
How stressed has the user been?
How many times did the user consume high glycemic foods?
Did the user experience any skin sensitivity (e.g., redness, peeling, and itching)?
Does the user wish to provide any data considered relevant to skin health?

For example, if the responses to the initial set of questions or images are indicative of a condition including blackheads or whiteheads, a determination can be made regarding which topical combination is ideal for the treatment of the condition. Lesion count may also provide a basis for devising the treatment for the user.

In step 240, the program 105 determines if the treatment plan is effective at treating the skin conditions of the user. In addition to this explicit data, supplemental information and insight into a user's health includes data integrated from various sensors and applications on device 107 that measure fitness, health, hormonal cycle, diet, stress, and so forth. The responses to the questions give a broader picture of the user's health and habits. Some habits, such as face washing are critical to overall acne development, making this information relevant to skin health. It is also important to know if users are following the treatment plan, such as using the suggested products as directed or regularly using the suggest products.

Upon receiving feedback and a recommended course of action or treatment, the user can track their treatment and outcome via user-generated logs. The user-generated logs are comprised of questions designed to determine the efficacy and progression of the treatment and photographs. This documents the progression of the skin condition and treatment.

Based on the answers provided in the skin care log, the program 105 generates feedback that is personalized to the user. As users continually update how their skin health and habits are changing, a neural network is used by the program 105 to learn over time the weighted influence of all of the data points on outcome, which is the effectiveness of the treatment plan.

In step 250, if the program 105 determines the treatment plan is effective at, for example, reducing acne or skin anomalies, then the generated treatment plan has been validated as an optimal treatment for reducing the acne or other skin anomalies. This validation result is sent to the database 120 and may further train the AI-model used by the program 105 in generating and devising a treatment plan.

In step 245, if the program 105 determines the treatment plan is effective at, for example, reducing acne or skin anomalies, then the generated treatment plan is modified. The program 105 then performs step 235 to monitor the progress the modified treatment plan, as depicted in FIG. 2. The program 105 needs to refine the models and algorithms applied to obtain an optimized treatment plan. As the data continues to grow and the model continues to be trained, the program 105 learns over time what optimal treatment and feedback each user subset should receive. The possible categories and clustering of users may expand over time as the program 105 becomes more intelligent in estimating outcome based on changes in treatment and behavior. As this is occurring over time, feedback may change and be customized to every user. Weighted together with the explicit feedback, the program 105 may further use this information to understand and improve user experience. Additionally, the program 105 may devise a new treatment plan by tracking the size of inflammation; determining if there is any reduction in inflammation; or observing changes in number of acne regions on the user's face. By analyzing sequencing results and bacteria rations that integrated into the program 105, the treatment plan can be altered depending on severity of imbalance.

Below are a few scenarios demonstrating modification of a user's feedback and treatment, based on the feedback provided by the user.

In Scenario 1, the user has started on treatment b. After a week, the user has reported that she is not improving. It has been determined by the program 105 that the user has not been using products as directed and has not been washing her face. The program 105 provides the recommendation for Scenario 1 that treatment should not change yet, and that education and further coaching to the user should be provided. The education is directed to the importance of compliance in treatment and the importance of face washing.

In Scenario 2, the user has started on treatment c. After a week, the user has reported that she is improving slightly, but her skin is feeling dry and slightly irritated. The program 105 recommends for Scenario 2 that the user change application of the product on skin to once daily instead of twice daily. The user's next treatment will be changed to treatment b, made for individuals with more sensitive skin.

In Scenario 3, the user has started on treatment c. After a week, the user reports that she is improving slightly, but is still suffering from some acne. She reports there is no skin sensitivity or irritation. She is following her treatment regimen of the treatment pan exactly as suggested by the program 105, including face washing regularly. The program 105 recommends for Scenario 3 that the treatment plan can be altered to a slightly stronger formulation (treatment d). The users applying treatment d do not exhibit irritation or any significant improvement. An alternate formulation to treatment c or treatment d may help in achieving desired results. Stated another way, the program 105 may devise a combination of treatment products/compositions that may improve the skin health of the user.

In Scenario 4, the user has skin discoloration. Based on the hue, texture, and intensity of different regions of the skin of the user, the program 105 determines the magnitude of improvement and if the different regions are closer in color in comparison to prior to the treatment. A threshold parameter for the infected region, which is bleached compared to other regions of the skin, has to increase in intensity and hue by at least 30% in two weeks. If treatment a does not increase the intensity and hue to at least 30% within two weeks, then the program 105 recommends in Scenario 4 that the user modify treatment regimen a to treatment b. However, if the threshold was met, then the program 105 suggests to the user that he or she stay with treatment regiment b.

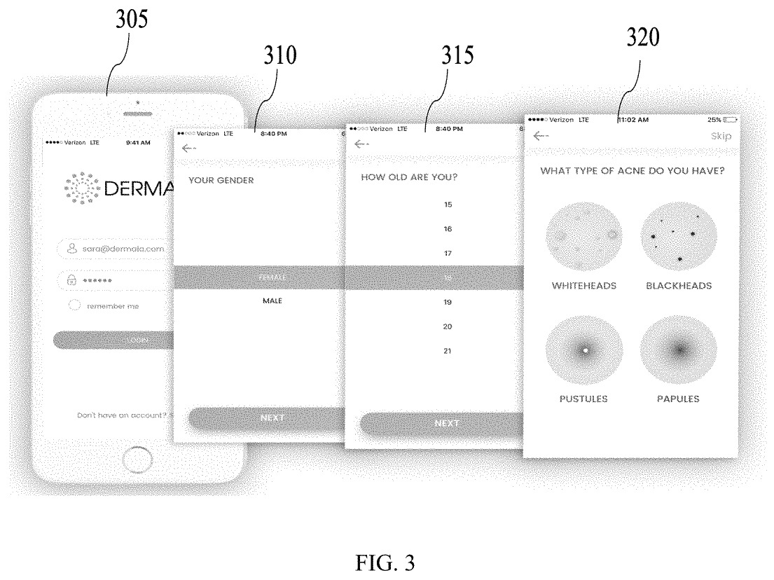

FIG. 3 is an example of users signing up for a service supported by the program 105, the users are prompted to enter information, as depicted in GUIs 305, 310, 315, and 320. In the GUI 305, the user activates an instance of the service supported by the program 105. To login and use the service, the user must enter his or her user identification (e.g., an email address or personalized handle) and password protected by encryption in the GUI 305. The program 105 extracts predetermined questions from the database 120 as a set of initial set of questions for the user to respond to, including gender in the GUI 310, age in the GUI 315, and type of acne (e.g., whiteheads, blackheads, pustules, and papules) from which they suffer in the GUI 320.

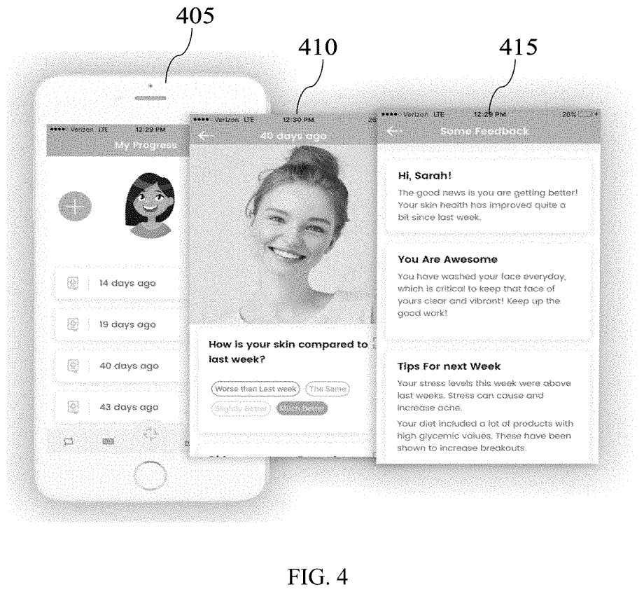

In FIG. 4, GUIs 405, 410, and 415 are progress tracking screens which may be maintained by the program 115. A user may view previous logs in the profile (as described with respect to FIG. 2), as depicted in the GUI 405; create new logs in the GUI 410; and read the generated feedback in the GUI 415.

To create a log, users may upload a photo for the program 115 to receive, and provide response to questions surrounding their skin health and skin care regimen, as depicted in the GUI 410. The program 115 may extract designated questions from database 120 or derive new questions, based on prior responses and image correlations.

Based on a current image processed and analyzed by the program 115, current feedback, and prior feedback, customized and personalized feedback is generated and presented to the user, as depicted in the GUI 415.

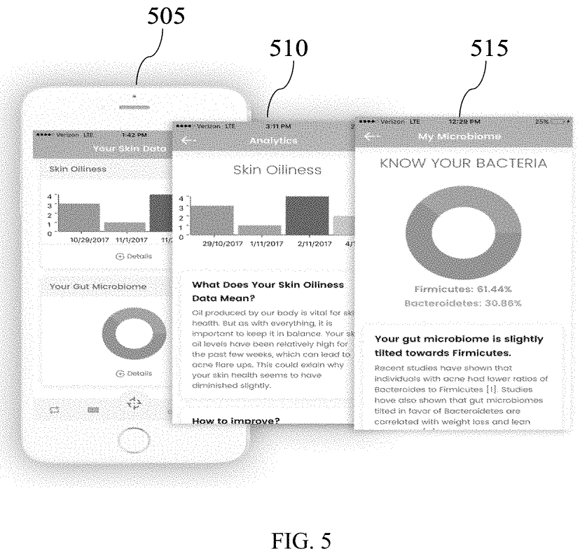

In FIG. 5, a user tracks his or her skin characteristics over time. The program 105 applies AI-techniques, as described above, to identify which changes are related to the skin health of the user. The program 105 may request microbiome sequences, genome sequences, epigenome sequences, or pH readings. Samples may be collected via swab and other collections methods known in the art. The obtained sequencing data (e.g., microbiome, genome, epigenome, and so forth) may be integrated into the database 120 by the program 105.

In one example, if microbiome sequencing is requested, the program 105 may connect to a vendor and instruct the vendor to send a package for microbiome sequencing. Upon receiving the page, the user may collect a sample for microbiome sequencing. The results of the microbiome sequencing are analyzed by the program 105 and may be beneficial as an additional data stream. The additional data stream analyzed by the program 105 yields a more detailed insight into factors and/or events impacting the skin of the user. The program 105 applies data analytics show the results of skin oiliness and gut microbiome sequencing, as depicted in GUI 505. The data analytics can build meaningful connections between user skin health and daily/weekly actions.

Based on existing knowledge of certain species and strains of bacteria, the program 105 can alter a customized treatment plan, such as the treatment regimen or treatment compositions, to rebalance the bacterial makeup. For example, the program 105 may use the results of the microbiome in the gut in conjunction with skin oiliness. As an AI-supported digital application, the program 105 has learning capabilities. Thus, acne as a condition may be further investigated using deep learning techniques. The deep learning techniques may suggest or determine how certain changes in skin health characteristics relate to the microbiome data. Based on the data used to derive the GUI 505 and deep learning techniques applied on the contents of database 120, which has, for example, medical disclosures directed skin oiliness and Firmicutes, the program 120 may output a rationalization, explanation, and/or implication of the output in the GUI 505. In GUI 510, an explanation of the skin oiliness data from the GUI 505 is provided. In GUI 515, an explanation of the gut microbiome data from the GUI 505 is provided, which indicates the user's “gut microbiome is slightly tilted towards Firmicutes.” As the data provided by users expands, the program 105 may further expand the treatment capabilities at an individual level. For example, the customized treatment plan suggests that the user take supplements or alter ingredients entirely, based on the needs of the user's microbiome.

By validating models that correctly determine how certain skin health characteristics change and refining models that incorrectly determine how certain skin health characteristics change, this could: (i) provide the basis for a customized treatment to improve skin health, as depicted on the bottom portion of the GUI 510; and/or (ii) further improve upon the customized treatments. More specifically, the program 105 determines trends among users with similar and different microbiome sequences to identify new factors that may impact skin health. Similarly, trends among users with similar and different genome, proteome, epigenome, metabolome, and so forth may be used by the program 105 to identify new factors that may impact skin health.

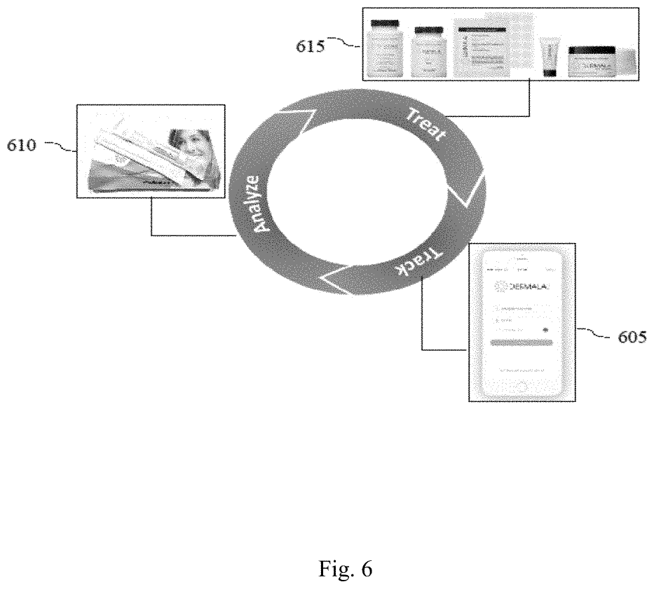

The loop in FIG. 6 illustrates the continuous updates for a devised treatment plan where the program 105 facilitates the user to track treatment progress in step 605, based on: (i) analyzed image and integrated data (e.g., microbiome, genome, proteome, epigenome, and metabolome sequences) in step 610; and (ii) received treatment recommendations in step 615. The program 105 learns and corrects itself, based on: user feedback and skin health progression; the responses to questions; and user characteristics compiled in the profile. Based on a score assigned to the user, the user is matched with a treatment regimen and treatment compositions. The score is determined by a skin health questionnaire asked in “My Progress” section, and user characteristics may be determined using the responses to the registration questionnaire. This data is sent to the server 120 for processing and instant feedback from the program 105. The algorithms applied the program 115 take the instant feedback and any data previously stored in the database 120 for the user for analysis. The program 105 may query the database 120 for information on users with similar characteristics; community-based recommendations; and updates to user feedback.

Based on the data from the integrated sensor or applications and the responses to the questions (i.e., Q1-Q3 in FIG. 7), the users are placed into one of n number of groups (i.e. groups a-h in FIG. 7). The program 105 may obtain images of skin; responses to questions; and data from integrated sensors and applications that measure fitness, health, hormonal cycles, diet, stress, and so forth. For example, a set of users give responses indicating that they suffer predominantly from blackheads, with 5-10 pimples per week and have sensitive skin. The set of users could be placed into group b. The set of users associated with group b are recommended personal treatment b. The program 105 begins to apply the rule-based classification for clustering users as depicted in FIG. 7.

As the dataset of users evolves and their resulting feedback expands, the dataset may train a model used by the program 105, based on deep learning techniques. The program 105 can be a recommender system using a collaborative filtering algorithm based on deep neural networks to devise and suggest customized treatment plans to each user among a group of users. Over time, the program 105 may learn similarities and differences among users and recommend the treatment plans that are most likely to work for users with similar characteristics.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So for example performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y, and Z to obtain the benefit of such steps. Thus method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

### EXAMPLES

Aspects of the present teachings may be further understood in light of the following examples, which should not be construed as limiting the scope of the present teachings in any way.

### Example 1—Acne Tracker App

The present invention includes methods and a computer system for tracking and optimizing treatment of skin diseases, and in particular, inflammatory acne vulgaris. The program 105 utilizes data analytics and machine learning to track users' acne and optimize treatment.

The Acne Tracker App is a variant of the program 105 that may be an application installed on a mobile device such as a mobile phone, tablet, laptop, or other device, or may be entirely web-based and accessible through a web browser on a mobile device, laptop, desktop computer, or other device. Once the user obtains the Acne Tracker App or accesses the web-based application, the user registers with the application by providing the required personal or identifying information. The user is then prompted to answer a few questions about themselves and their skin health, as well as take a picture or photograph of the affected area. The questions may include without limitation those pertaining to age, gender, severity of acne, acne lesion count, type of acne, skin type, skin sensitivity, known allergies, and/or previous treatment history. The questions and related data may be stored on the device on which the application is being executed or may be downloaded from a server hosting the system (e.g., the server 115) and other data (e.g., the database 120). The picture or photograph may be captured by the user utilizing the camera connected to or incorporated with the device on which the Acne Tracker App is being executed or the web-based application is being accessed. An image that is captured by a separate camera or device may also be uploaded via the Acne Tracker App or web-based application.

When a user is prompted to take a picture or photograph of the skin condition or affected area, the Acne Tracker App utilizes computer vision API's in combination with the device's camera or a camera connected to the device to determine the angle of the user's face, exposure, and other characteristics that are necessary for a high-quality image. The application may provide guidance to the user instructing the user to adjust lighting or face positioning by providing on-screen guidance. The pictures may then be stored on the non-transitory computer-readable medium of the device on which the application is being execute or accessed, as well as being uploaded via an internet connection to a server hosting the application, data, and other system components. This initial picture of the skin condition may serve as the initial metadata utilized to generate the first/initial recommendations for a treatment plan.

A number of methods have been deployed within the application to track and recommend treatment plans for users suffering from acne vulgaris. These methods include rule-based recommendations, face, and blemish detection algorithms, community-based recommendation systems, and bio statistics. Based on all of the data a user has logged, the application may recommend changes to a user's behavior, treatment regimen, or treatment compositions. Users of the application may record progress logs over time. These logs ask questions on a weekly basis, which can provide insight regarding the user's health. For example, the questions are designed to collect data pertaining to a user's skin health, product usage, hygiene, diet, stress, or hormonal activity. Such questions may include without limitation:

How is the user's skin compared to last week?

Has the user been using any DERMALA® products?

How often does the user wash his or her face?

How oily is the user's skin?

How stressed was the user this week?

How many times did the user eat high glycemic foods this week?

Each question is typically provided with four multiple-choice options, each having weighted scores. However, the questions may have more or fewer options, or may consist of binary yes/no responses. The questions being asked, as well as the available options and weight of the options, may also vary based on the progression of the treatment or other factors. When a user submits the logs, the scores are analyzed and recommendations are determined. For example, if a user has not been washing their face at the level recommended, the program 105 returns a recommendation for increasing frequency of face washing. The program 105 may examine historical data. If a user has been stressed at a high level for the last four weeks, the program 105 recommends employing techniques to reduce stress levels.

The system uses machine learning to perform acne detection and classification of a user-provided image. An image may be collected during signup or registration to use the application, as well as on daily, weekly, bi-weekly, monthly, or at other intervals as required for the user's condition or treatment, and is submitted with the periodic logs. This image is analyzed by the system to classify the type of acne as well as the severity of the condition. While the initial image analysis is used as a baseline comparison for the future records, the weekly logs and images allow for tracking of treatment outcome. When a user captures an image, the image is sent to a remotely located server. The server may be a physical device or virtual device existing in other infrastructure, such as the Amazon Web Services cloud. The images undergo processing and analysis, and are then stored in a file location or the database. Computer vision algorithms will analyze the image and perform lesion classification and count, as well as other characteristics associated with acne. This is done to analyze improvement of the condition and acne severity more objectively than may be captured by the questions or other user-provided data. In this example, the system may employ a U-Net convolutional neural network for lesion detection and count. By analyzing a user's image periodically, the system may recommend changes to treatment regimen or treatment composition, as well as provides engaging encouragement to the user.

Users may also be asked questions to provide demographic or historical information, to give the program 105 a better understanding of their skin health. Questions may include demographic information such as gender or age, and more detailed descriptions of the users' acne and duration of time they have been suffering. This data is used to further classify the user and generate community-based recommendations. The data and results obtained by users with similar characteristics are used to further refine the recommendation algorithms. Based on all questions a user answers throughout their use of the app, a user score is generated. This score is used to classify our users into subsets of user groups. Users within certain ranges will be recommended certain treatments. By collecting user characteristics, recommendations can be further refined by looking at what has worked for users with similar characteristics. For example, if user A suffered from moderate Acne, is female, and is 26 years old, she can be used as a reference for user B with similar characteristics. User B has just begun using the application, and it is beneficial to recommend an ideal treatment plan at the outset. If user A saw the most improvement using a treatment plan that includes kit X, user B is recommended to start with kit X, instead of the kit Y that user A has been recommended based on other criteria. The algorithms use collaborative filtering to build a recommendation system for the users. Collaborative filtering is based on grouping users having similar traits to the user the program 105 is trying to classify and understand. The application may also include a store section, allowing the user to connect to an e-shop to purchase the recommended treatments. Additionally, users may submit questions through the application to receive feedback from health professionals or other users.

### Example 2—Integration of Microbiome Analysis

While photos and user-generated feedback provide valuable and actionable insight, the program 105 further supports user prognosis and diagnosis by analyzing the gut, oral and skin microbiome and metabolome sequences. Users are sent a kit that collect microbiome samples by swabbing skin, stool, or saliva or using other method. Samples are analyzed for microbiome and metabolome composition, including without limitation ratio of beneficial to acne-causing bacteria and microbiome diversity levels. By analyzing and processing this data, the program 105 further pinpoints the dysbiosis occurring in a user's skin and connects the pinpointed finding to images displaying treatment outcomes and quantification data of the skin conditions. This allows the Acne Tracker application to recommend treatment plans that balances the dysbiosis in the user's skin and gut. After the program 105 recommends a treatment regimen, the user follows the treatment regimen for predetermined time, before re-sequencing is performed. By pinpointing the bacterial and state of the user's microbiome, effective treatments can be more accurately recommended, and the efficacy of recommended treatments can be tracked. For microbiome sequencing, certain bacteria ratios may be used to determine imbalance. For example, the gut microbiome sample is analyzed to determine the ratio of Firmicutes to Bacteroidetes. Recent studies have shown that users with acne had lower ratios of Bacteroidetes to Firmicutes. Depending on ratio, particular treatment plans can be recommended to users. If needed, these ratios can be monitored and probiotic treatments can be adjusted to increase the Bacteroidetes level.

The users are provided real time feedback on their skin health, as well as data visualizations that depict all relevant characteristics in their skin care. Users are able to track their stress levels, skin oiliness, product usage, overall skin health, and other vital skin health indicators. Correlations are built to show, for example, the link between stress and the user's current skin health state. Additionally providing a data understanding to the user provides a holistic view on skin health that has not previously been provided to users. As mentioned above, when users submit logs, they answer several multiple-choice questions. These questions have weighted scores that can be depicted graphically over time. For example, a user can see how their skin oiliness or stress level has changed has changed over the course of the last two months. These plots are displayed below the overall skin health plot. This allows users to see how changes in behavior, treatment regimen, or treatment compositions are altering their overall skin health over time. When a user selects a plot, they will be directed to a detailed page, that will explain the data point (e.g., skin oiliness, stress level, and so forth), and what impact it has on overall skin health. This provides education and insight into impacts (aside from treatment) of the user's lifestyle on acne prevalence. Users can be educated by their own improvement over time. Conventional methods of treatment do not provide this type of data and analysis to users.

### Example 3—Eczema Tracker App

Similarly as for acne vulgaris, the mobile app and data analytics platform and prediction algorithms used by the program 105 aid in diagnosis, prognosis, outcome tracking and treatment optimization for eczema, also known as atopic dermatitis. For eczema the platform would be the same, but the questions asked would be altered. For example, questions are focused on the affected area, instead of lesion count, and other data such as, is the affected area itchy, inflamed, flaking, etc. The feedback is specific to controlling eczema, and improving skin eczema.

### Example 4—Skin Health Tracker App

Similarly for acne vulgaris, the mobile app, the program 105 uses data analytics platform, and prediction algorithms to aid in diagnosis, prognosis, outcome tracking, and treatment optimization for skin aging and providing treatment to users with aging skin. Users are asked about wrinkles, dry skin, loose skin, product usage, and so forth. The application feedback would be specific to skin aging, and improving skin health.

### Example 5—Acne Reducing Using DERMALA® Acne Treatment Pads as a Devised Treatment

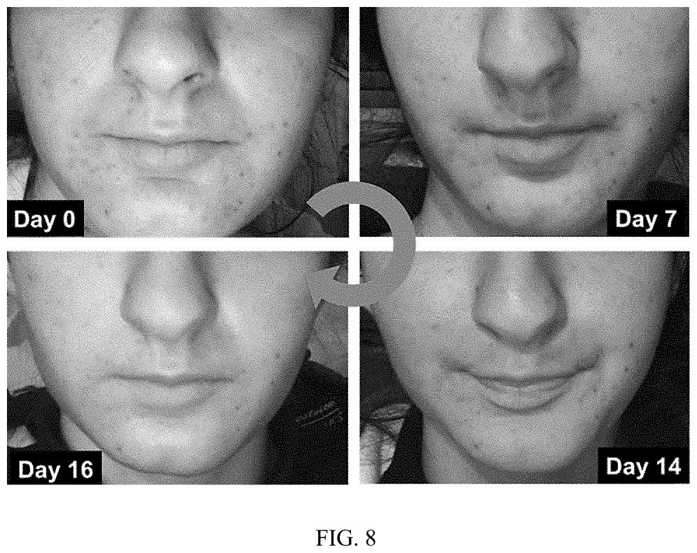

FIG. 8 is a series of images of a user that applied the generated or devised treatment plan. A mobile application, as supported by the program 105, aims to optimize treatment of a skin condition using the devised treatment plan. Within 16 days, the user achieves noticeable results. The user also exhibited gradual improvement at the 7 day and 14 day marks, as depicted in FIG. 8. The mobile application provides the user with consistent coaching and engagement to improve the efficacy of the treatment. After the first week's log, the user is coached to alter the treatment regimen to include twice a day exfoliation with DERMALA® Acne Treatment Pads, instead of once a day. The user is also encouraged to use the DERMALA® cleanser regularly as the user was not using that product. These changes in conjunction with continued good habits, led to a rapid improvement for the user.

### Example 6—Devising a Treatment Plan for a User Based on Analysis of Another User

A user named Sarah has signed up for the digital service supported by the program 105 and reported that she is a 19-year-old female living in Brooklyn, N.Y. She has provided responses to the initial classification questions and uploaded an image. These provide explicit data points to initially classify her, such as age; gender; and location (e.g., environmental factors may impact skin health). She has reported that she suffers from whiteheads weekly and has approximately 10 whiteheads at any given time. Based on the image she has provided, the program 105 validates that she has moderate acne and can track the regions in which she suffers. We have found that some of these factors match Emma, who is another user of the service living in Los Angeles, Calif. Emma went through initial recommendations, while modifying the customized treatment plan presented to her. Treatment d is her most successful treatment plan. From Emma's results which are sent to the database 120, the program 105 may learn from Emma's history and directly recommend Sarah to start with treatment d. As more users like Emma and Sarah funnel through the service and the provided treatment plans, the program 105 adapts and learns what is ultimately working for user subsets based on several factors that can grow over time. However, if Sarah has sensitive skin in comparison to Emma, the program 105 may alter the frequency of which the treatments regimens should be applied. For example, the treatment regimen is suggested to be applied once a day, instead of twice a day. The personalization provided by the program 105 is not limited to recommendations directed to treatment regimes and treatment compositions. The program 105 may provide behavioral suggestions directed, but not limited, to: dietary recommendations; additional beauty routine recommendations; and recommendations on managing other aspects of a user's life (i.e., stress) in the treatment plan. The program 105 can further personalize and learn about environmental factors, such as weather and seasonal changes and how they relate to user's skin health. Harnessing this data could lead to changes in treatment regimens, treatment recommendations, and other treatment recommendations, based on the season and weather a user's hometown is experiencing. With respect to Sarah and Emma, the weather conditions of Brooklyn, N.Y. and Los Angeles, Calif. are factors used to devise the treatment plan, analyzing the progress of the treatment plan, and modifying the treatment plan.

### Example 7—Gamification as a Method to Identify New Variable and Enhance the Capability of the AI-Supported Digital Application

The program 105 prompts users to complete and provide additional data for a complete skin health profile in the form of a virtual game. Accompanying discounts and rewards for adding logs and providing additional feedback may be provided. The AI capabilities of the program 105 are used to build a valuable multi-dimensional dataset by generating a virtual game. Aside from learning from users which treatments are working best for them and improving on the suggested and customized treatment plans, the program 105 introduces new questions and new methods for data collection, in order to uncover different factors and how they relate to overall skin health. Deep learning is used to uncover variables and their respective weights/impacts on skin health relating to acne or other chronic skin health conditions.

The user participates in “sprints” generated by the program 105, which suggests specific changes to the user's habits for a defined period of time. Examples of these changes may be directed to: diet via elimination of specific foods (e.g., milk, sugar, etc.); behavior via hygienic mannerisms (e.g., face washing, hygiene after exercising); product usage; or cosmetic or physical appearance by eliminating makeup, skincare products, hair conditioner, wearing a cap, and so forth.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which does not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A method for treating and monitoring skin conditions for each user of one or more users, the method comprising:
receiving medical information of each user of the one or more users;
receiving demographic information of each user of the one or more users;
receiving one or more images of each user of the one or more users, wherein the one or more images depict one or more affected skin areas;
sending the medical information for each user of the one or more users, the demographic information for each user of the one or more users, and the one or more images for each user of the one or more users to a server;
generating an artificial intelligence (AI)-supported model to yield a profile for each user of the one or more users, at the server, wherein the profile for each of the one or more users comprises the medical information, the demographic information, and the one or more images;
extracting contents from a database using the AI-supported model, wherein the contents from the database comprise established treatment plans, medical findings, and effects of local environments on skin regions;
applying the AI-supported model on the profile for each of the one or more users and the contents from the database, and thereby:
establishing a baseline for each user of the one or more users, and
devising a customized plan for each user of the one or more users;
receiving subsequent medical information for each user of the one or more users and subsequent one or more images of the one or more users;
comparing the subsequent medical information for each user of the one or more users and subsequent one or more images of the one or more users to the medical information for each user of the one or more users, the demographic information for each user of the one or more users, and the one or more images for each user of the one or more users, and thereby tracking progress of the customized plan of each user of the one or more users; and
outputting effectiveness of the customized treatment plan for each user of the one or more users.

2. The method of claim 1, wherein the medical information and the subsequent medical information for each user of the one or more users comprise a type of acne and location of the type of acne.

3. The method of claim 1, wherein the demographic information and the subsequent medical information for each user of the one or more users comprise age, gender, and location for each user of the one or more users.

4. The method of claim 1, wherein generating the AI-based model comprises:
treating the one or more images and the subsequent one or more images of each user of the one or more users and the contents as a first data stream; and
treating the medical information and the subsequent medical information of each user of the one or more users and the demographic information of each of the one of the one or more users as a second data stream.

5. The method of claim 1, wherein tracking progress of the customized plan for each user of the one or more users comprises:
identifying a first set of factors impacting the one or more regions of the skin;
and
adding a second set of factors to the database.

6. The method of claim 1, further comprising:
requesting biological test results; and
integrating the biological test results into the database.

7. The method of claim 1, wherein devising the customized treatment plan for each user of the one or more users, comprises:
receiving inputs to inquiries from each user of the one or more users;
comparing the inputs to the inquiries from each user of the one or more users;
identifying similarities and differences between the inputs to the inquiries from each user of the one or more users;
grouping the one or more users as at least a first set and a second set, based on the similarities and differences.

8. The method of claim 7, wherein grouping the one or more users as at least the first set and the second set comprises:
devising a customized treatment plan for the first set; and
devising a customized treatment plan for the second set.

9. The method of claim 1, further comprising:
generating a virtual game based on the AI-based model.

10. The method of claim 6, wherein the biological tests are used to obtain microbiome, genome, epigenome, and pH data.

11. The method of claim 1, wherein outputting the effectiveness of the customized treatment plan for each user of the one or more users, comprises:
determining if the effectiveness of the customized treatment plan for each user of the one or more users meets a threshold level;
validating a customized plan as effective if the effectiveness is above the threshold level; and
modifying the customized plan if the effectiveness is below the threshold level.

12. A computer program product comprising computer executable code embodied in a non-transitory computer readable medium that, when executing on one or more computing devices, performs steps of:
receiving medical information of each user of one or more users;
receiving demographic information of each user of the one or more users;
receiving one or more images of each user of the one or more users, wherein the one or more images depict one or more affected skin areas;
sending the medical information for each user of the one or more users, the demographic information for each user of the one or more users, and the one or more images for each user of the one or more users to a server;
generating an artificial intelligence (AI)-supported model to yield a profile for each user of the one or more users, at the server, wherein the profile for each of the one or more users comprises the medical information, the demographic information, and the one or more images;
extracting contents from a database using the AI-supported model, wherein the contents from the database comprise established treatment plans, medical findings, and effects of local environments on skin regions;
applying the AI-supported model on the profile for each of the one or more users and the contents from the database, and thereby:
establishing a baseline for each user of the one or more users, and
devising a customized plan for each user of the one or more users;
receiving subsequent medical information for each user of the one or more users and subsequent one or more images of the one or more users;
comparing the subsequent medical information for each user of the one or more users and subsequent one or more images of the one or more users to the medical information for each user of the one or more users, the demographic information for each user of the one or more users, and the one or more images for each user of the one or more users, and thereby tracking progress of the customized plan of each user of the one or more users; and
outputting effectiveness of the customized treatment plan for each user of the one or more users.

13. The computer program product of claim 12, wherein the medical information and the subsequent medical information for each user of the one or more users comprise a type of acne and location of the type of acne.

14. The computer program product of claim 12, wherein the demographic information and the subsequent medical information for each user of the one or more users comprise age, gender, and location for each user of the one or more users.

15. The computer program product of claim 12, wherein generating the AI-based model comprises:
treating the one or more images and the subsequent one or more images of each user of the one or more users and the contents as a first data stream; and
treating the medical information and the subsequent medical information of each user of the one or more users and the demographic information of each of the one of the one or more users as a second data stream.

16. The computer program product of claim 12, wherein tracking progress of the customized plan for each user of the one or more users comprises:
identifying a first set of factors impacting the one or more regions of the skin; and
adding a second set of factors to the database.

17. A system comprising:
a server;
a computing device in communication with the server over a network, the computing device including a processor and a memory, the memory bearing computer executable code configured to perform the steps of:
receiving medical information of each user of the one or more users;
receiving demographic information of each user of the one or more users;
receiving one or more images of each user of the one or more users, wherein the one or more images depict one or more affected skin areas;
sending the medical information for each user of the one or more users, the demographic information for each user of the one or more users, and the one or more images for each user of the one or more users to a server;
generating an artificial intelligence (AI)-supported model to yield a profile for each user of the one or more users, at the server, wherein the profile for each of the one or more users comprises the medical information, the demographic information, and the one or more images;
extracting contents from a database using the AI-supported model, wherein the contents from the database comprise established treatment plans, medical findings, and effects of local environments on skin regions;
applying the AI-supported model on the profile for each of the one or more users and the contents from the database, and thereby:
establishing a baseline for each user of the one or more users, and
devising a customized plan for each user of the one or more users;
receiving subsequent medical information for each user of the one or more users and subsequent one or more images of the one or more users;
comparing the subsequent medical information for each user of the one or more users and subsequent one or more images of the one or more users to the medical information for each user of the one or more users, the demographic information for each user of the one or more users, and the one or more images for each user of the one or more users, and thereby tracking progress of the customized plan of each user of the one or more users; and
outputting effectiveness of the customized treatment plan for each user of the one or more users.

18. The system of claim 17, wherein the medical information and the subsequent medical information for each user of the one or more users comprise a type of acne and location of the type of acne.

19. The system of claim 17, wherein the demographic information and the subsequent medical information for each user of the one or more users comprise age, gender, and location for each user of the one or more users.

20. The system of claim 17, wherein generating the AI-based model comprises:
treating the one or more images and the subsequent one or more images of each user of the one or more users and the contents as a first data stream; and
treating the medical information and the subsequent medical information of each user of the one or more users and the demographic information of each of the one of the one or more users as a second data stream.
